(19) United States
(12) Patent Application Publication
(10) Pub. No.: US 2012/0330390 A1
(43) Pub. Date: Dec. 27, 2012

(54) Medical devices and implants from Ta-Nb-W alloys

(75) Inventors: Jürgen Wachter, Rodermark (DE); Jens Trötzschel, Neuwiedermus (DE)

(73) Assignee: HERAEUS PRECIOUS METALS GMBH & CO. KG, Hanau (DE)

(21) Appl. No.: 13/480,922

(22) Filed: May 25, 2012

Related U.S. Application Data
(63) Continuation of application No. 11/804,029, filed on May 16, 2007.
Application No. 11/804,029 is a continuation-in-part of application No. 10/409,559, filed on Apr. 8, 2003, now abandoned.
Application No. 10/409,559 is a continuation of application No. 11/804,029, filed on May 16, 2007.
Application No. 11/804,029 is a continuation-in-part of application No. 10/409,559, filed on Apr. 8, 2003, now abandoned.
Application No. 10/409,559 is a continuation-in-part of application No. 12/070,646, filed on Feb. 19, 2008.
Application No. 12/070,646 is a continuation of application No. 10/409,559, filed on Apr. 8, 2003, now abandoned.
Application No. 10/409,559 is a continuation of application No. 12/717,425, filed on Mar. 4, 2010.
Application No. 12/717,425 is a continuation of application No. 12/070,646, filed on Feb. 19, 2008.

(30) Foreign Application Priority Data
Feb. 10, 2003 (EP) 03 002 905.2

(51) Int. Cl. A61N 1/05; B32B 15/04; C22C 27/02

(52) U.S. Cl. 607/116; 420/427; 148/422; 148/317

(57) ABSTRACT

The present invention relates to a medical device or implant made at least in part of a high-strength, low-modulus metal alloy comprising niobium, tantalum, and at least one element selected from the group consisting of zirconium, tungsten, and molybdenum. The medical devices according to the present invention provide superior characteristics with regard to biocompatibility, radio-opacity and MRI compatibility.

### CROSS-REFERENCE TO RELATED APPLICATIONS

This application is a continuation of U.S. patent application Ser. No. 11/804,029, filed May 16, 2007, which is a continuation-in-part of U.S. patent application Ser. No. 10/409,559, filed Apr. 8, 2003, now abandoned. This application is also related to U.S. patent application Ser. No. 11/804,044, filed May 16, 2007, now abandoned, which was a continuation-in-part of Ser. No. 10/409,559; Ser. No. 11/804,040, filed May 16, 2007, now abandoned, which was a division of Ser. No. 10/409,559; Ser. No. 12/070,646, filed Feb. 19, 2008, now allowed, which is a continuation of Ser. No. 10/409,559; and Ser. No. 12/717,425, filed Mar. 4, 2010, now allowed, which is a continuation of Ser. No. 12/070,646. The present application therefore also claims priority from Ser. No. 12/070,646 and Ser. No. 12/717,425 as a continuation-in-part. All of these applications claim ultimate priority from European patent application No. 03 002 905.2, filed Feb. 10, 2003. The entire contents of the preceding applications are incorporated herein by reference.

### BACKGROUND OF THE INVENTION

The present invention relates to improved metal alloys for medical implants or devices for desired material properties.

A medical implant or device must satisfy a number of requirements. Factors affecting the choice of the medical implant or device and the material thereof are mainly all mechanical properties and biocompatibility. The material must not cause any inflammatory reaction or allergic reaction. Commonly used materials often include nickel, like medical grade 316L stainless steel, which contains about 16% nickel. For patients with an allergic reaction the implantation of such materials is contraindicated. Another consideration in material selection is the need for the implanting physician to be able to visualize the position of the medical implant or device during the procedure to the desired target site in the body, and for purposes of examination from time to time thereafter at the implant site, typically by X-ray fluoroscopy.

With the growing importance of magnetic resonance imaging (MRI), MRI compatibility is desirable. The metal alloys commonly used for implantation (like stainless steel 316) induce a local disturbance of the magnetic field used in MRI, to the extent that imaging of surrounding tissue is impeded. Although alloys like Nitinol™ behave more favorably in MRI, their MRI compatibility is not considered to be sufficiently good.

This invention also relates to medical devices or implants in general, such as catheters, guide wires, stents, stent grafts, and heart valve repair devices.

Stents are generally thin-walled tubular-shaped devices composed of complex patterns of inter-connecting struts which function to hold open a segment of a blood vessel or other body lumen like esophagus and urethra. Stent grafts are stents with a circumferential covering or lining and are suitable for supporting a dissected artery or intimal flap that can occlude a vessel lumen. Stents and stent grafts are typically implanted by use of a catheter. Initially they are maintained in a radially compressed state to maneuver them through the lumen. Once in position, they are deployed. The material from which the vascular prosthesis like stents or stent grafts is constructed must allow the prosthesis to undergo expansion, which typically requires substantial deformation. Once expanded the stent must maintain its size and shape and must be capable of withstanding the structural loads, namely radial compressive forces, imposed on the stent as it supports the walls of a vessel lumen. The wall of the prosthesis must be sufficiently thick, depending on the stent material, not only to withstand the vessel wall recoil but also allow the stent to be seen on the fluoroscope. Finally, the prosthesis material must be biocompatible, so as not to trigger any adverse vascular responses like re-stenosis or thrombus formation in the treated vessel.

For medical devices, such as all kind of catheters and guide wires, special mechanical properties are desired to have perfect trackability and pushability during the intervention. Moreover, good radio-opacity and MRI compatibility are essential in order to survey medical procedures via X-ray and MRI. Finally, also for these medical devices biocompatibility is a must.

In the past years increased effort was undertaken to find new materials for medical implants and devices bearing superior characteristics over commonly used metals like stainless steel or titanium. Numerous publications focus on titanium alloys aiming at corrosion-resistant, high-strength and biocompatible alloys. As described, for example, in U.S. Pat. No. 6,312,455, U.S. patent application Publication No. 2001/0007953 A1, and International patent application Publication No. WO 99/58184 A1, many titanium alloys thereof are super-elastic or shape memory alloys. A pseudo-elastic β-titanium alloy fabricated from titanium, molybdenum, aluminum, and optionally niobium, chrome and vanadium is described in U.S. Pat. No. 6,258,182. European Patent No. EP 0 788 802 provides a self-expanding stent consisting of a titanium alloy including at least about 68 weight percent titanium and optionally niobium, zirconium, and molybdenum. U.S. Pat. No. 6,238,491 and International patent application Publication No. WO 00/68448 A1 describe a niobium-titanium-zirconium-molybdenum alloy for medical devices providing a uniform β-structure, which is corrosion-resistant, and can be processed to develop high strength and low modulus. The alloy comprises 29 to 70 weight percent niobium, 10 to 46 weight percent zirconium, 3 to 15 weight percent molybdenum, and a balance of titanium.

In another approach, Davidson (European patent application Publication No. EP 0 601 804 A1) employs an alloy consisting essentially of titanium, 10 to 20 or 25 to 50 weight percent niobium, and optionally up to 20 weight percent zirconium, the alloy having an elastic modulus less than 90 GPa. Similar titanium alloys for medical implants also published by Davidson comprise titanium, 10 to 20 or 35 to 50 weight percent niobium, and optionally up to 20 weight percent each zirconium and tantalum (European Patent No. EP 0 437 079) or titanium, 10 to 20 or 35 to 50 weight percent each niobium and tantalum, and optionally up to 20 weight percent zirconium (U.S. Pat. No. 5,690,670). European patent application Publication No. EP 0 707 085 A1 also provides a low-modulus, biocompatible titanium-based alloy for medical devices consisting of 20 to 40 weight percent niobium, 4.5 to 25 weight percent tantalum, 2.5 to 13 weight percent zirconium, and the balance titanium. A further high-strength, low-modulus and biocompatible titanium alloy is laid open in U.S. Pat. No. 4,857,269 and European patent application Publication No. EP 0 359 446 A1 consisting of titanium and up to 25 weight percent niobium, zirconium, and molybdenum. European patent application Publication No. EP 1 046 722 A1 describes a corrosion-resistant titanium-zirconium-type alloy for medical appliances consisting of 25 to 50 weight percent titanium, 5 to 30 weight percent niobium, 5 to 40 weight percent tantalum, and 25 to 60 weight percent zirconium.

Further approaches to develop biocompatible, high-strength alloys, which are also sufficiently radio-opaque and do not contain titanium, are described in U.S. Pat. No. 6,478,815 and International patent application Publication No. WO 02/43787 A1. Both documents reveal stents made from at least 90 weight percent niobium. Niobium is a relatively soft and ductile metal, which is alloyed with traces of other elements, e.g. zirconium, tantalum or titanium for reinforcement of the alloy. However, niobium surfaces cannot be electropolished because of their tendency to smear. Stents fabricated from binary tantalum alloys, namely tantalum-niobium and tantalum-tungsten, are disclosed in International patent application Publication No. WO 02/05863 A1.

### BRIEF SUMMARY OF THE INVENTION

An aim of the present invention is to provide an inventive material for medical implants and devices, which comprises favorable mechanical properties, excellent biocompatibility, optimal radio-opacity while at the same time exhibiting minor image artifact in MRI examination (MRI compatibility), and does therefore overcome the drawbacks of recently available metals for medical purposes.

The alloy fulfils all mechanical and structural requirements according to its function in a medical implant or device. Moreover, the device is sufficiently radio-opaque to allow for good imaging of the device under X-ray without the addition of an extra layer or portion of radio-opaque material. Also, the device is not overly bright, and therefore does not obscure the image of the surrounding tissue, as would be the case with a device made from an extremely dense material. In addition, the device is MRI safe and compatible, preferably also visible under MRI.

Surprisingly, it has been found that the desired properties can be given to a metal alloy comprising tantalum, niobium, and at least one element selected from the group consisting of tungsten, zirconium and molybdenum.

### DETAILED DESCRIPTION OF THE INVENTION

Tantalum is known as a very hard metal with a high melting point, high strength, and good ductility and is almost completely inert at body temperature. Tantalum has a high atomic number (73) and a density of 16.6 g/cm³ resulting in a high radio-opacity. Therefore, medical implants or devices made of pure tantalum have the disadvantage that they are excessively radio-opaque, leading to a completely black area on the X-ray image in the region where the medical implant or device is located.

The radio-opacity of the inventive metal alloys is adjusted by adding further elements possessing higher or lower atomic numbers to the tantalum based alloy, which lowers the density of the alloy. Niobium has an atomic mass of approximately half that of tantalum. Thus, tailoring the density of the inventive alloy by variation of the niobium portion allows achievement of appropriate radio-opacity for each medical device or implant manufactured at least in part of the inventive alloy. It is possible to fabricate an alloy according to the present invention, which is sufficiently radio-opaque to be readily visualized under X-ray during medical procedures and yet is not so radio-opaque as to interfere with the visualization of surrounding body tissue.

The alloys of the invention show excellent melting and mixing properties with excellent uniformity, since niobium and tantalum are arbitrarily miscible. Varying the amount of tungsten, zirconium, and molybdenum, or optionally, the amount of cerium, rhenium, or hafnium, allows adjustment of the granular size of the alloy.

Surprisingly, the alloy according to the present invention is stronger than pure tantalum and in specific compositions even stronger than stainless steel. In a preferred embodiment a stent is manufactured from the alloy of the invention comprising a tailored radio-opacity while having a reduced wall thickness. Such a stent combines desired visibility under X-ray and excellent radial force with minimized delivery profile and less turbulence when employed in the vessel.

An additional advantage of the inventive alloy is the formation of a passive oxide film primarily composed of tantalum-oxide (Ta₂O₅), which is generally more durable and more corrosion-resistant than, for example, the chromium-oxide film formed during the passivation of stainless steel.

The inventive alloy can be easily cold-worked to increase strength and reduce elastic modulus. It is possible to form a hard, abrasion-resistant surface on the inventive alloy through standard oxidation and nitriding methods known by those skilled in the art. The presence of a hard, inert, abrasion-resistant surface layer presents an important option for medical implants and devices in which it is desirable to have lower friction and wear, electrical insulation and improved corrosion resistance.

To further improve the biocompatibility of the medical implant or device fabricated at least in part from the inventive alloy, at least a portion of the surface of the inventive alloy can be conversion surface-hardened and/or coated. Such coatings can include, but are not limited to a polymer, a blend of polymers, a metal, a blend of metals, a ceramic, and/or biomolecules, in particular peptides, proteins, lipids, carbohydrates, and/or nucleic acids (e.g. collagen, heparin, fibrin, phosphorylcholine, cellulose, morphogenic proteins or peptides, growth factors). Furthermore, the alloy surface or the coatings can comprise stem cells and/or bioactive substances, in particular drugs, antibiotics, growth factors, anti-inflammatory agents, and/or anti-thrombogenic agents. Further, the surface can be modified by electropolishing or mechanical polishing for formation of a completely smooth surface, sintering to achieve a porous coating, as for example described in EP 0 601 804 A1, or by roughening procedures or micro-blasting, in particular sand-blasting, to achieve a rough surface.

The inventive alloy is useful in the manufacturing of a variety of medical implants and devices. The manufacture of medical devices from the inventive alloy includes minimal-invasive devices, in particular guide wires, catheters (balloon catheters, guiding catheters, angiographic catheters, functional catheters, etc.), intra-cavernous implants, in particular intra-esophagus, intra-urethra, intra-tracheal implants, and intra-vascular implants, in particular stents, stent grafts, stent graft connectors, heart valve repair devices, or filters.

Preferred alloys contain the following elements:

(a) between about 0.1 and 70 weight percent Niobium,

(b) between about 0.1 and 30 weight percent in total of at least one element selected from the group consisting of tungsten, zirconium, and molybdenum,

(c) up to 5 weight percent in total of at least one element selected from the group consisting of hafnium, rhenium and lanthanides, in particular cerium,

(d) and a balance of tantalum.

The alloys preferably provide for a uniform beta structure, which is uniform and corrosion-resistant, and have the ability for conversion oxidation or nitriding surface-hardening of the medical implant or device.

The tungsten content is preferably between 0.1 and 15 weight percent.

The zirconium content is preferably between 0.1 and 10 weight percent.

The molybdenum content is preferably between 0.1 and 20 weight percent and more preferably between 0.1 and 10 weight percent.

The niobium content is preferably between 5 and 25 weight percent.

Especially preferred alloys contain about 10 weight percent niobium and about 2.5 weight percent tungsten.

Also preferred are alloys which comprise about 10 weight percent niobium and about 7.5 weight percent tungsten.

Also preferred are alloys which comprise about 10 weight percent niobium and about 1 weight percent zirconium.

Also preferred are alloys which comprise about 10 weight percent niobium and about 3 weight percent zirconium.

The invention also relates to medical implants or devices fabricated from the above-mentioned alloys, e.g. minimal-invasive devices, in particular catheters or guide wires, or intra-cavernous implants, in particular intravascular implants, such as stents, stent grafts, stent graft connectors, or heart valve repair devices.

In the above implants and devices the surface of the metal alloys may be passivated by oxidation or nitriding, or may be electropolished, mechanically polished, micro-blasted, roughened or sintered, or may be coated with a polymer, a blend of polymers, a metal, a blend of metals, a ceramic, and/or biomolecules, in particular peptides, proteins, lipids, carbohydrates, and/or nucleic acids; or may be coated with stem cells and/or a bioactive substance, in particular drugs, antibiotics, growth factors, anti-inflammatory agents, and/or anti-thrombogenic agents.

### EXAMPLES

The invention may be carried out with an alloy of the following compositions:

1. Ta: 71.5% by weight
Nb: 27.5% by weight
Zr: 1.0% by weight

2. Ta: 82.5% by weight
Nb: 10% by weight
W: 7.5% by weight

3. Ta: 87% by weight

Nb: 10% by weight
Mo: 3% by weight

4. Ta: 83% by weight
Nb: 10% by weight
Mo: 7% by weight

Methods of producing the alloys are known to the person skilled in the art.

1. A medical device comprising a biocompatible structure configured for implantation or medical intervention in a human body, the medical device excluding intravascular implants, wherein the medical device comprises components at least partially fabricated from a tantalum-based metal alloy, the tantalum-based metal alloy comprising:
about 10 wt % niobium;
about 7.5 wt % tungsten; and
the balance tantalum.

2. The medical device according to claim 1, wherein the tantalum-based metal alloy further comprises:
0.1 to 10 wt % zirconium.

3. The medical device according to claim 1, wherein the tantalum-based metal alloy further comprises:
about 1 wt % zirconium.

4. The medical device according to claim 1, which is a minimal-invasive device.

5. The medical device according to claim 1, which is one of a catheter, a guide wire, an intra-cavernous implant, a heart repair device, and a filter.

6. The medical device according to claim 1, which is one of a stimulation device, a stimulation lead, and a dental device.

7. The medical device according to claim 1, wherein tantalum-based metal alloy further comprises up to 5 wt % in total of at least one element selected from the group consisting of hafnium, rhenium and a lanthanide.

8. The medical device according to claim 7, wherein the lanthanide is cerium.

9.-12. (canceled)

13. The medical device according to claim 1, wherein the tantalum-based metal alloy has a surface that is passivated by oxidation or nitriding.

14. The medical device according to claim 1, wherein the titanium-based metal alloy has a surface that is one of electropolished, mechanically polished, micro-blasted, roughened, and sintered.
